(19) United States
(12) Patent Application Publication
(10) Pub. No.: US 2026/0210861 A1
(43) Pub. Date: Jul. 23, 2026

(54) STROBOSCOPIC HIGH-THROUGHPUT IMAGING SYSTEM

(71) Applicant: Staker Scientific, Inc., Livermore, CA (US)

(72) Inventor: Bryan Staker, Livermore, CA (US)

(21) Appl. No.: 19/456,592

(22) Filed: Jan. 22, 2026

Related U.S. Application Data
(60) Provisional application No. 63/748,649, filed on Jan. 23, 2025.

(51) Int. Cl. G01N 21/64

(52) U.S. Cl. CPC G01N 21/6486; G01N 21/6458; G01N 2201/0697; G01N 2201/1087

(57) ABSTRACT

A fluorescence imaging system is configured for imaging a plurality of fluorescent biomolecules in a sample wherein the sample may be an array of fluorescent biomolecules. The system acquires a plurality of sequentially offset, overlapping, smeared sub-images of the sample wherein all of the sub-images are obtained with the array of fluorescent biomolecules maintained in a common state across all the sub-images, and estimates a distance between sub-images by correlating pairs of sub-images. The system assembles and averages the sub-images to obtain improved SNR in a final image.

### CROSS-REFERENCE TO RELATED APPLICATIONS

The present application claims priority to U.S. Provisional Application No. 63/748,649, filed on Jan. 23, 2025, and titled “High-Throughput Imaging System,” the entirety of which is incorporated by reference herein.

### BACKGROUND

The field of biomolecule imaging has progressed rapidly over the last two decades. For example, biomolecule arrays may be imaged for DNA or RNA sequencing, DNA or RNA identification, protein sequencing, protein identification, etc. In DNA or RNA sequencing, conventional high-throughput systems process 1,000 giga-bases or higher per day of sequencing data. But such systems typically require heavy vibration isolation systems made of steel or granite with complex staging systems. Moreover, the staging systems often utilize high quality and high cost linear or air bearing stages that are expensive and heavy. It is common for such systems to weigh at least 200 to 500 and higher kilograms with potential high capital costs. Such conventional systems inhibit or prevent vibration by using heavy, precise mechanical stages. The systems capture each part of a final image once, with an exposure time long enough to ensure adequate signal-to-noise ratio (SNR) to obtain results usable for subsequent analysis.

In addition, conventional systems prevent image smear by using a time-delay integration (TDI) camera and/or an optical image stabilization system. This is done so that there is no relative motion between the object and the camera image sensor during an image exposure. In such systems, image exposure time is sufficiently long to collect enough light to achieve acceptable signal-to-noise ratio in one exposure.

### SUMMARY

In view of the foregoing, there is a need for an efficient high-throughput imaging system that achieves comparable performance of conventional high-throughput systems but with reduced mass. In a non-limiting example, an efficient high-throughput imaging system has a mass of around 25 kilograms or less or around 50 kg-75 kg in another implementation. Disclosed is an optical system configured to obtain extremely high throughput rates such as those demanded by high-throughput imaging applications including DNA sequencing, but at a cost of less than 5% or 10% of typical systems.

In an example implementation, the optical system is part of a nucleic acid sequencing system for detecting a plurality of fluorescent biomolecules in a sample with a scanning fluorescence microscope. The system includes a sample stage configured to support the sample, wherein the sample stage is in motion with respect to a digital camera. The disclosed system has lightweight, and therefore only moderately effective, provisions for vibration isolation. Prior art systems go to great lengths (and use heavy weights) to prevent vibrations from affecting their cameras. The disclosed system uses fast (i.e. short) exposure times to mitigate the effects of vibration. If an exposure is short enough, vibrations do not generate image blur because the system doesn't move much during the exposure.

Unlike the prior art systems, the disclosed system does not use mechanical (e.g., scanning mirrors) or electronic (e.g., time-delay integration) techniques to compensate for the relative motion between the object and the image sensor. The short exposure times used in the disclosed system freeze out the effects of random vibrations (“blur” as described below.) The system intentionally allows smear of as much as about 1.1 times to about 4 times the width of the point spread function of the collection optics.

The digital camera of the system is optically coupled to the sample stage and configured to acquire a plurality of digital sub-images of the fluorescent biomolecules. Sub-images of the plurality of sub-images are spatially aligned and then averaged together to form a final image. In an implementation, the systems obtain usable images of one or more biomolecule arrays as part of a biomolecule array sequencing operation. A biomolecule array can be, but is not limited to, a rectangular array, a hexagonal array or a random array. An object to be imaged or scanned pursuant to biomolecule array sequencing scanning may have an area of about 2,000 mm² or 40 mm by 50 mm. There are billions of features within that area that need to be imaged.

As the sample stage moves with respect to the digital camera at a velocity ‘vS’, features in the resultant images may have smear. The system is configured to intentionally tolerate image smear. In this regard, the system comprises an optical system having a point-spread function (PSF) wherein a width W of the PSF is λ/(2 NA) (NA is the numerical aperture of the optical system.) The function may be a Gaussian function or some other function. The amount of smear in a sub-image is given by the relative velocity vS of the object and the image sensor of the camera multiplied by the exposure time tX. Thus:

$$smear = ( vS ) \cdot ( tX )$$

In the disclosed system, smear is greater than the width of the PSF by a factor of k, which is multiplied by the PSF. Thus:

$$smear = ( k ) \cdot ( Width of the PSF )$$

The factor k can be between about 1.5 and about 3. In another implementation, the k factor can be between 1.2 and 4 or 1.2 and 5. In an implementation, k=2. In an implementation, k=3 or k=4. In an implementation, k=1.3 or 1.4. In another implementation, k>1.05. The factor k does not need to be an integer and cannot be less than 1. In prior-art systems, k=1 or may be slightly more than 1, e.g. 1<k<1.05. In the disclosed system, k>1 such that the system tolerates an intentional loss of optical resolution unlike the prior art.

In another example implementation, there is disclosed a method of detecting plurality of fluorescent biomolecules in a sample supported on a sample stage, the method comprising: acquiring a plurality of digital images of the plurality of fluorescent biomolecules in the sample with a digital camera optically coupled to the to the sample stage, wherein the sample stage is in motion with respect to a digital camera during the acquiring; and averaging the plurality of digital images to form a final image thereby detecting the plurality of fluorescent biomolecules in the sample. The acquiring is at a rate from 500 frames per second (fps) to 5,000 fps and at an exposure time of 5-200 microseconds in non-limiting examples. The exposure time can vary. In another implementation, the acquiring is at a rate from 5,000 to 50,000 fps.

In one aspect, there is disclosed a fluorescence imaging system for imaging a plurality of fluorescent biomolecules in a sample, the system comprising: a sample stage configured to support the sample, wherein the sample is an array of fluorescent biomolecules; an optical system comprising a digital camera optically coupled to the sample stage, wherein the sample stage is in motion with respect to a digital camera; a scanning fluorescence microscope coupled to the camera; an illumination system that provides pulsed illumination; a non-transitory memory storing executable instructions that, when executed by a processor, cause the system to: acquire, by the digital camera, a plurality of sequentially offset, overlapping, smeared sub-images of the sample wherein all of the sub-images are obtained with the biomolecule array maintained in a common state across all the sub-images; estimate a distance between sub-images by correlating pairs of sub-images; and assemble and average the sub-images to obtain improved SNR in a final image.

In another aspect, there is disclosed a biomolecule array imaging system comprising: a flow cell configured to contain an array of fluorescent biomolecules; a camera, including an image sensor directly connected to dedicated processing hardware; a pulsed illumination system; optics, including a microscope objective lens, a tube lens and a dichroic mirror; wherein, the flow cell is mounted on a translation stage that moves the flow cell in a direction of motion with respect to the optics; while the stage moves the flow cell, the pulsed illumination system illuminates a sub-section of the flow cell via the optics, and the camera captures a first set of overlapping sub-images of the flow cell via the optics; the pulsed illumination system sets an exposure time for the sub-images; the dedicated processing hardware estimates a distance between pairs of sub-images by cross-correlating the pairs of sub-images; and the dedicated processing hardware aligns and averages sub-images to form a final image.

The details of one or more variations of the subject matter described herein are set forth in the accompanying drawings and the description below. Other features and advantages of the subject matter described herein will be apparent from the description and drawings, and from the claims.

### BRIEF DESCRIPTION OF THE DRAWINGS

FIG. 1 shows a schematic representation of a fluorescent imaging system for high throughput biomolecule array imaging.

FIG. 2 shows a diagram illustrating an embodiment of a general method for obtaining and processing images for high throughput biomolecule array imaging.

FIG. 3 shows a diagram illustrating an embodiment of a method for obtaining and processing images for high throughput biomolecule array imaging.

FIGS. 4-9 provide schematic representations of the processes of the method of FIG. 3.

FIG. 10 schematically illustrates an implementation of a data processing aspect of the system

FIG. 11 depicts a block diagram illustrating an example of a computing system consistent with implementations of the current subject matter.

### DETAILED DESCRIPTION

Before the present subject matter is further described, it is to be understood that this subject matter described herein is not limited to particular embodiments described, as such may of course vary. It is also to be understood that the terminology used herein is for the purpose of describing particular embodiments only, and is not intended to be limiting. Unless defined otherwise, all technical terms used herein have the same meaning as commonly understood by one skilled in the art to which this subject matter belongs.

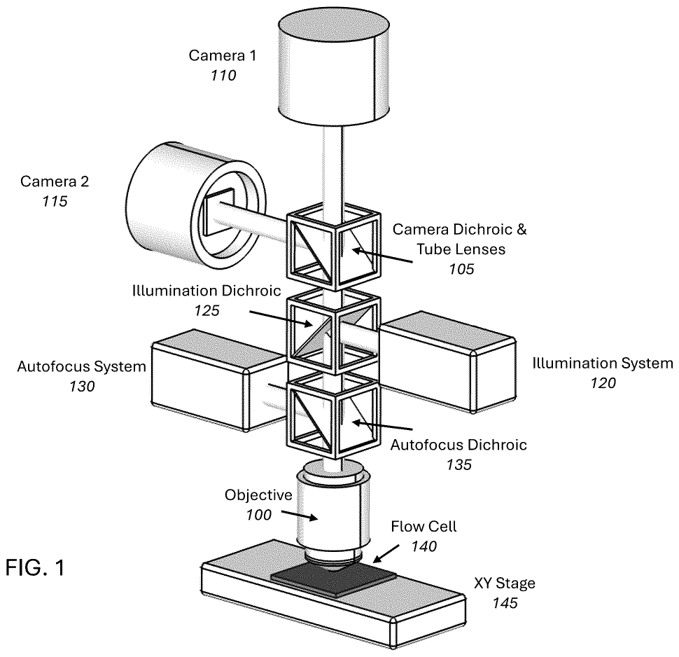

FIG. 1 shows a non-limiting, schematic representation of a fluorescent imaging system such as a system configured for high throughput biomolecule array sequencing. The system is configured to obtain a plurality of sub-images of an object, such as a biomolecule array, for collectively forming a final image of the object. The sub-images comprise multiple short exposures that traverse across a larger imaging area wherein the sub-images are not stabilized or tracked with respect to the movement of the object such that the sub-images may have smear. However, the sub-images due not have blur as short exposure time eliminates blur from being present in the sub-images. All of the sub-images (for forming a first final image) are obtained with the biomolecule array maintained in a common state across all the sub-images.

The system averages the sub-images to obtain a final image that is collectively formed of the sub-images. Traditional systems eliminate smear by tracking, using either time-delay integration cameras or image tracking and stabilizing optics. The disclosed system eliminates the use of image tracking and stabilization optics and allows for smear in resultant sub-images as the stage moves and vibrates relative to the imaging system. As used herein, “blur” in an image is a reduction in image sharpness due to random x-, y-, and z-direction, relative movement between the camera and the imaged object during an exposure. This may be, for example, due to vibration of the object or a platform coupled to the object. In the prior art, blur may be prevented by reducing the amplitude and frequency of random x-, y-, and z-direction motion. The prior art thus attempts to prevent blur by reducing the amplitude and frequency of random x-, y-, and z-direction motion. The disclosed system prevents blur by making the image exposure duration so short that the object doesn't sufficiently move during exposure time range of the image.

As used herein, “smear” for an image is a reduction in image sharpness in one direction only (such as an x-direction) such as due to intentional, relative motion between the camera and the object. It should be appreciated that conventional systems have no net smear or reduce the smear to a negligible amount such as below 10% or a factor k of 1.1. In the disclosed system, images are intentionally obtained with smear. The amount of smear in an image is the relative speed of the object and the image sensor multiplied by the exposure time.

The disclosed system thus does not attempt to compensate for relative motion between the object and the camera image sensor during image exposure. The image exposure time is short enough to eliminate blur caused by system vibrations although not necessarily short enough to eliminate smear caused by relative motion between the moving stage and stationary camera image sensor. A consequence with such a short exposure time is that not enough light is collected during each exposure to achieve acceptable signal-to-noise ratio. The disclosed system builds up signal-to-noise by adding up many sub-images, wherein each sub-image is offset, or shifted compared to a prior obtained sub-image. They are shifted because the object is continuously moving. It moves between the time of one exposure and the next exposure.

With reference still to FIG. 1, the system includes an objective lens 100 and camera dichroic mirror and tube lens assembly 105 and one or more camera(s) 110 and 115 configured to image a sample on a flow cell 140 positioned on a movable XY sample stage 145. The flow cell 140 contains a fluorescently tagged biomolecule array, which can be a DNA array. The cameras 110 have image sensors that record sub-images of the flow cell. The sub-images are focused onto the image sensors by microscope optics of the system. The microscope optics are characterized by a point spread function (PSF). The point spread function has a width. The system can further include a dichroic mirror 105 if needed for multiple cameras. The system includes an illumination system 120 with illumination dichroic mirror 125, and an autofocus sub-system 130 with dichroic filter 135. The XY stage is configured to move relative to the camera(s) 110 and 115 as the cameras obtain images. A temperature control system can be thermally coupled to the sample stage The system further includes a Z-stage for autofocus as well as a fluidics and reagent systems. In an implementation, a sub-image covers 225 μm by 900 μm of a flow cell in the description. Approximately that area can be fit into different width/length aspect ratios.

The camera may have an output data rate greater than 100 billion bits per second (Gb/s). In an implementation, the output data rate is 300 Gb/s. To accommodate such high data rates, the camera can include an image sensor and an initial processing chip which are integrated together on the same chip, system-on-a-chip, interposer, chip package or circuit board. The initial processing chip can be, in non-limiting example, a field programmable gate array (FPGA), graphics processing unit (GPU), other custom application specific integrated circuit (ASIC), or a chip specifically designed for the image assembly task. Such chips process the sub-images (described herein) and build up the final image in a pipelined process.

In an implementation, the system does not have a time-delay integration camera and/or does not have an optical image stabilization system. In an implementation, the camera, optics and sample stage collectively weigh less than 100 kg.

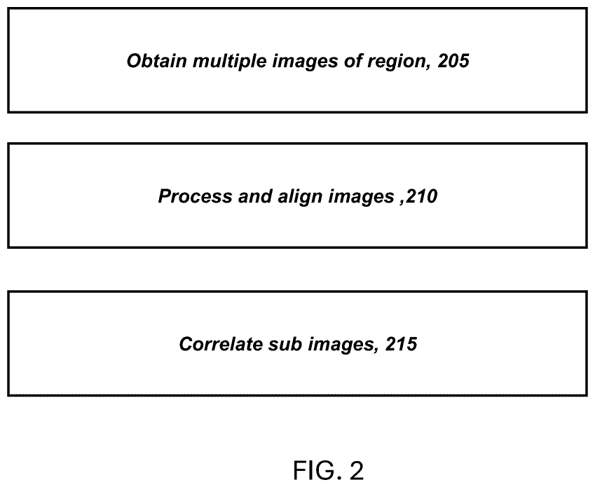

FIG. 2 shows a diagram illustrating an embodiment of a general method for obtaining and processing images for high throughput biomolecule array sequencing. As discussed, the system obtains multiple sub-images of a biomolecule array, wherein all of the sub-images (for forming a first final image) are obtained with the biomolecule array maintained in a common state across all the sub-images. There is no biochemical processing of the biomolecule array between each sub-image. The method includes one or more example operations 205-215. The method can be implemented by one or more computer processors of the system and may include greater or fewer operations than illustrated in FIG. 2. The operations can further be performed in an order different than that illustrated in FIG. 2. In addition, one or more operations of the system can optionally perform with interaction of a human.

In operation 205, the camera of the system obtains multiple sub-images (wherein each sub-image can be, as examples, about 100 μm by 800 μm, about 200 μm by 800 μm, or about 400 μm by 800 μm) of a flow cell as the flow cell moves relative to the camera. An obtained plurality of sequentially offset, overlapping, smeared sub-images may be averaged together to form a final image usable for later analysis. In a set of sub-images, each sub-image is offset from the previous one by a small quantity of pixels, e.g. 20-50 based on the relative velocity between the camera and the flow cell. Each sub-image may overlap the next sub-image by, e.g., 50% to 99% of the sub-image area. In the final image, each pixel is derived from an average of N sub-images where N may vary and in a non-limiting implementation is about 2 to about 100. The system may discard one or more sub-images as soon as they have been assembled into the final image. For example, a first sub-image may be discarded long before the last sub-image is acquired by the image sensor. In an implementation, all the sub-images are discarded.

The flow cell is positioned on an X-Y stage and moves via movement of the X-Y stage. The system does not and need not synchronize the stage with the camera while the stage moves relative to the camera. The number of sub-images taken can vary. In a non-limiting example, the system takes between 20 to 200 sub-images of the region although the quantity can vary. In another example implementation, the system takes 50 separate sub-images of the region to achieve an ensemble of sub-images. Each of the sub-images of the ensemble are combined, such as by averaging the sub-images of the ensemble, to form a final image.

The relative movement between the fixed position camera and the moving stage results in the sub-images potentially having a smear that corresponds to the direction of velocity of the stage. The amount of smear may be calculated by the velocity of the stage multiplied by the exposure time of the sub-image.

The resultant sub-images are unaffected by vibration. This is because the images are taken at frequencies that are above or far above resonance for common modes which can be easily constrained to be below 200 Hz, even for light weight desktop systems. In a non-limiting example, the sub-images are taken at a rate on the order of 5 kHz with 35 microsecond exposure times, which may vary. In another implementation, the exposure time is 40 microseconds, 45 microseconds, or 50 microseconds, which are non-limiting. Any vibration of the system may cause the location of the sub-images to oscillate, but each given sub-image will be free of vibration blur.

The system thus obtains the multiple sub-images at rates far above step and shoot systems (which are typically at 10 fps) or the fastest TDI and image stabilized systems (which are typically at 100 fps-200 fps). As an example, a two-camera system, with each camera operating at a 5 kHz frame rate (5,000 fps), acquires 10,000 sub-images per second. These are example distinguishing factors relative to prior systems although other distinguishing factors are within the scope of this disclosure.

In operation 210, the system processes and aligns the sub-images to form the final image. In an implementation, the system aligns the sub-images by first identifying a small region of each sub-image, such as a 256×256 region in a non-limiting example. The size of each region can be selected to obtain data that defines the average distance between sub-images. The sub-images are thus selected with an estimated offset so that the sub-images are very similar. Any residual shifts between images are then a result of low frequency vibrational noise in the system or errors in stage positions. Each image will be taken at an exposure time that is a small fraction of the effective exposure time of all the images combined (i.e. 2% for the case of 50 sub-images).

In a next operation 215, two or more of the sub-images are correlated to determine lateral shift deviation from ideal. The regions defined by the sub-images are mostly overlapping (such as by about 99%.) The correlation need not be between consecutive images. It may be between images that are a few exposures apart. The images being correlated contain some overlapping points. The correlation may be between small patches of sub-images rather than between complete sub-images.

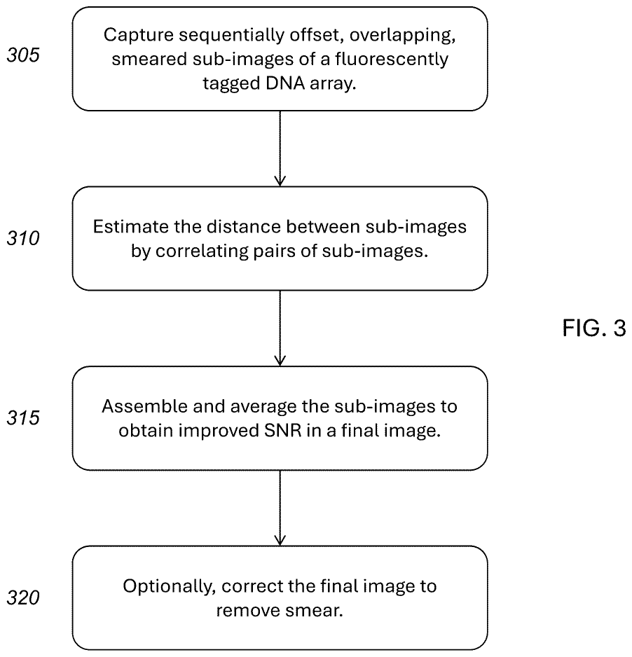

FIG. 3 shows a diagram illustrating an embodiment of a method with additional details for obtaining and processing images for high throughput biomolecule array sequencing. The method includes one or more example operations 305-320. The method can be implemented by one or more computer processors of the system and may include greater or fewer operations than illustrated in FIG. 3. The operations can further be performed in an order different than that illustrated in FIG. 3. In addition, one or more operations of the system can optionally perform with interaction of a human.

In operation 305, the camera captures offset, overlapping, smeared sub-images of at least a portion of an object. In an implementation, the object is a fluorescently tagged biomolecule array. All of the sub-images (for forming a first final image) are obtained with the biomolecule array maintained in a common state across all the sub-images. Thus, there is no biochemical processing of the biomolecule array between each sub-image. The obtained sub-images are subsequently used to form at least a first final image. After biochemical processing of the biomolecule array, additional sets of sub-images of the biomolecule array in a different biochemical state may be obtained for forming additional final images.

Each sub-image is offset along an axis with another sub-images due to relative movement between the camera and the object. The sub-images are thus offset relative to one another along a directional axis over a length of the biomolecule array defined by the directional axis. A sub-image is overlapping with another sub-image when a feature or landmark of the object on one image is also contained in the other sub-image. The object may move fast enough relative to the camera that features or landmarks in the sub-images may be smeared in the direction of motion.

The system captures several sequentially offset, overlapping, smeared sub-images, each of which may have a poor signal-to-noise ratio (SNR). In an implementation, the SNR of each sub-image is 5× to 100× lower or 10× to 100× lower than required for later analysis. The sub-image capture is immune to blur as the exposure time is much less than the inverse of the highest frequency problematic vibration of the system. In an implementation, the exposure time is 10-20 times less than the inverse of the highest frequency problematic vibration of the system.

Each sub-image is formed by light collected during a short exposure time. The exposure time is set by a short illumination pulse. The illumination system 120 provides pulsed illumination as short illumination pulses having a duration between about 10 μs and about 100 μs. The illumination pulses are synchronized to occur when the camera shutter is open. The illumination may be provided by a pulsed laser, such as a diode laser or a diode-pumped solid-state laser, or by a continuous laser modulated by an optoelectronic or mechanical shutter. The pulse repetition rate is more than 1 kHz. In one implementation the illumination pulses are about 50 μs long and occur with a repetition rate of about 4 kHz, synchronized with a camera having a 4 kHz frame rate. The system may be described as “stroboscopic” because it uses pulsed illumination. The pulsed illumination system illuminates a sub-section of the flow cell via the microscope optics.

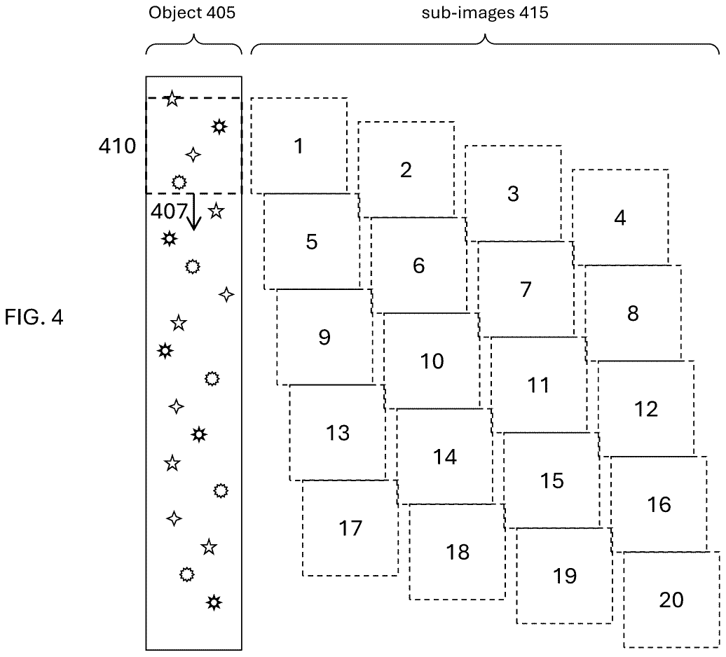

FIGS. 4-6 schematically represent the operation 305. FIG. 4 shows a schematic representation of an object 405, which may be for example a fluorescently tagged biomolecule array positioned on a mechanically movable stage. The object 405 and the stage move along an axis 407. As the object 405 moves relative to an image frame 410 of the camera, the camera obtains a series of successive sub-images 415 of a region of the object 405 within the image frame 410. The sub-image are obtained at a predetermined rate. The movement of the object 405 along the axis 407 results in relative movement between the image frame 410, which is in a fixed position, and the object 405. In another implementation, such relative movement is achieved via movement of the image frame 410 and a fixed position object 405, or by movement of both the image frame 410 and the object 405.

As the object 405 moves, a plurality of sub-images 410 of the corresponding region of the object 405 are obtained with the sub-images being sequentially offset from one another along the axis 405. Thus, there is an offset distance between each sub-image. In FIG. 4, the sub-images are labeled as sub-images 1-20 although the quantity of sub-images may vary. It should be appreciated that the quantity of 20 sub-images is merely an example for purposes of simple explanation and is not limiting.

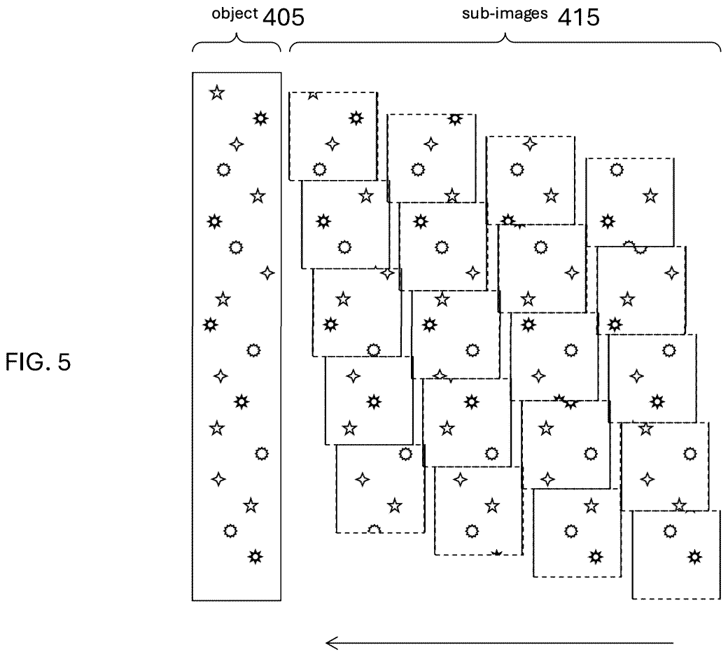

FIG. 5 shows the sub-images 415 populated with the corresponding region of the object 405 that the camera obtained as the object 405 moved along the axis 407 relative to the image frame. Note that the sub-images include sequentially spaced regions of the object 415 with consecutive sub-images including one or more common features or landmarks of the object 415 within a prior sub-image. Such landmarks may have smear in the corresponding image. As mentioned, the sub-images are immune to blur (which results from vibration) because the exposure time is much less (e.g. 10-20 times less) than the inverse of the highest frequency problematic vibration.

As mentioned, the system averages the offset, overlapping sub-images to obtain improved SNR in a final image. Once the offset between sub-images is known and removed, the sub-images can be averaged together to form a final image of the object 405. Averaging the sub-images depends on knowing the offset, or distance travelled, between sub-images. In operation 310, the system estimates the offset between sub-images by cross-correlating pairs of sub-images. The estimated offset is then used to adjust a relative position of two or more sub-images such that common, but offset, landmarks between the sub-images can be lined up with one another in a final image. The sub-images to be cross correlated may be consecutive with a prior sub-image or separated by several intermediate sub-images.

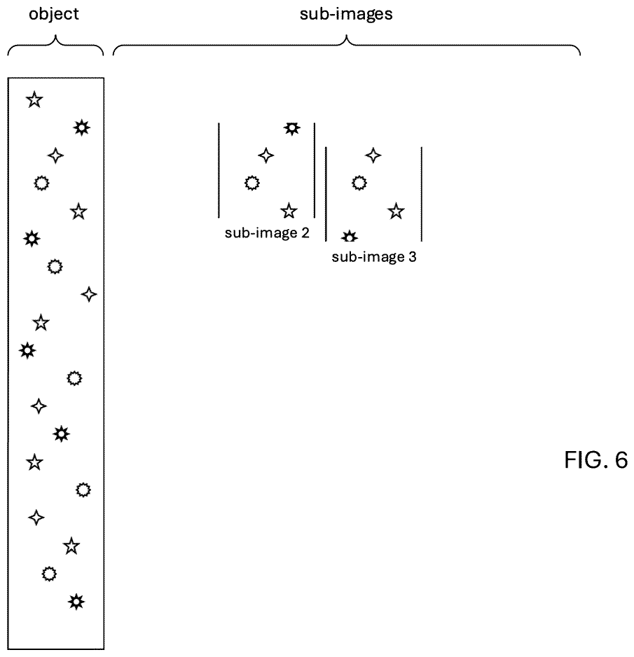

Cross correlation measures the similarity between two sub-images (such as between sub-image 2 and sub-image 3 in FIG. 6) as a function of the displacement, or offset distance, of one sub-image relative to the other sub-image. If there is no displacement, then the cross correlation is maximum. If the images aren't the same, then the cross correlation is low or zero, even if there is no displacement. Cross correlation thus provides a sharp signal where the relative displacement between sub-images is equal to the offset between the sub-images. As mentioned, the correlation need not be between consecutive sub-images. It may be between images that are a few exposures apart. The images being correlated must contain some overlapping landmarks or points, however.

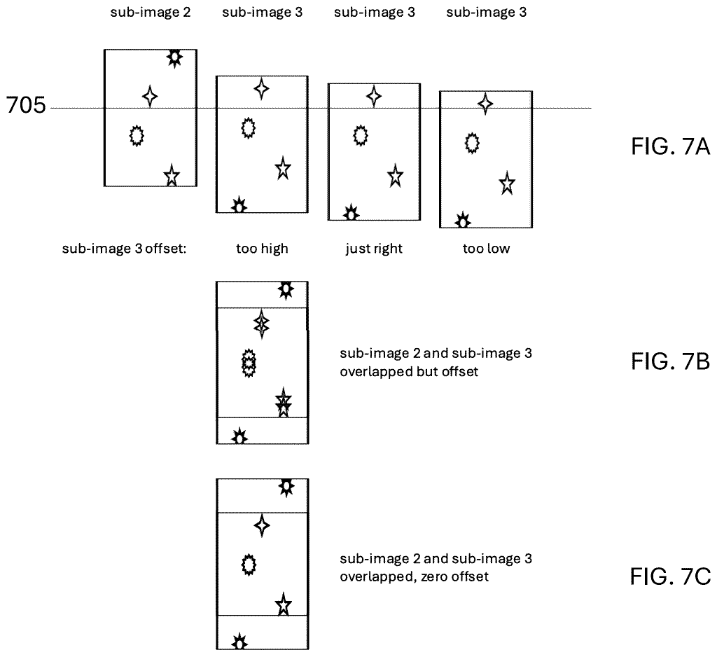

FIGS. 6 and 7A-7C schematically represent this process. FIG. 6 shows an example with sub-image 2 and a consecutive sub-image 3, each of which contain images of common, but offset, landmarks or features of the object. FIGS. 7A-7C schematically illustrates the process of lining up sub-image 2 and sub-image 3 such that the common features align. FIG. 7A shows an instance of sub-image 2 with an alignment line 705 that provides a visual gauge as to the offset of common landmarks between sub-image 2 and sub-image 3. In one instance, sub-image 3 is “too high” relative to sub-image 3 such that the common landmarks in sub-image 3 are offset too high above the common landmarks of sub-image 2. In another instance, sub-image 3 is “too low” relative to sub-image 3 such that the common landmarks in sub-image 3 are offset too low below the common landmarks of sub-image 2. FIG. 7B shows the sub-images overlayed such that the common landmarks are offset from one another. The overlay of FIG. 7B provides a visualization of how the landmarks of the object are offset from one another between sub-image 2 and sub-image 3.

With reference again to FIG. 7A, in another instance, the common landmarks are properly aligned “just right” such that the sub-images, when overlayed, provide properly overlaid, and aligned, landmarks. FIG. 7C shows the sub-images overlayed such that the landmarks have zero offset and are properly aligned with one another. Note that the landmarks have smear in each sub-image. As discussed below, such smear may optionally be corrected in the final image.

With reference again to FIG. 3, in operation 315, the system assembles and averages the offset, overlapping sub-images to obtain improved SNR in a subsequent final image. Once the system obtains or is otherwise provided with the offset distance between each sub-image, the system may spatially shift the sub-images to make them line up and then assemble the sub-images together to form the final image.

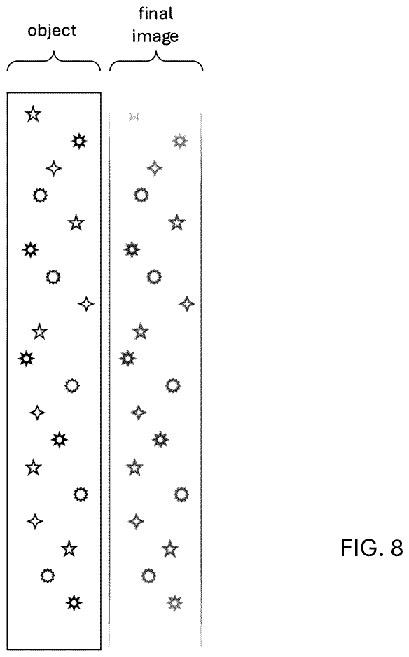

FIG. 8 schematically illustrates the object 405 and the corresponding, final image, which is comprised of the added-up sub-images. Each sub-image may be relatively dim and faint with a poor signal-to-noise ratio. In an implementation, a signal-to-noise ratio in one or more sub-images is not sufficient to permit extraction of usable information. In an implementation, the final image comprises usable images of fluorescently tagged biomolecule arrays as part of a biomolecule array sequencing operation.

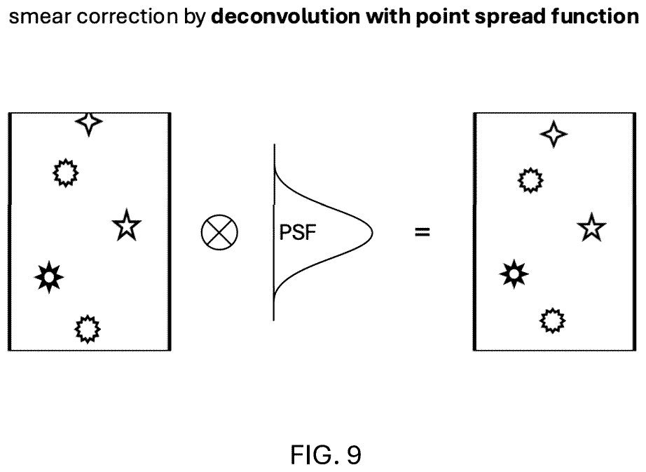

With reference again to FIG. 3, optionally in operation 320, the system corrects the final image to remove or otherwise correct smear. FIG. 9 schematically represents this process wherein the system uses the point spread function (PSF) to deconvolve the smeared final image (on the left of FIG. 9) to obtain a corrected, or non-smeared final image (on the right of FIG. 9). Removal or correction of smear is based on the speed of the stage during an exposure, the image exposure time, and the point spread function (impulse response) of the optical system. The speed may be estimated as described above. Smear correction is optional and may not be required if the amount of smear is not much larger than the features of interest in the final image.

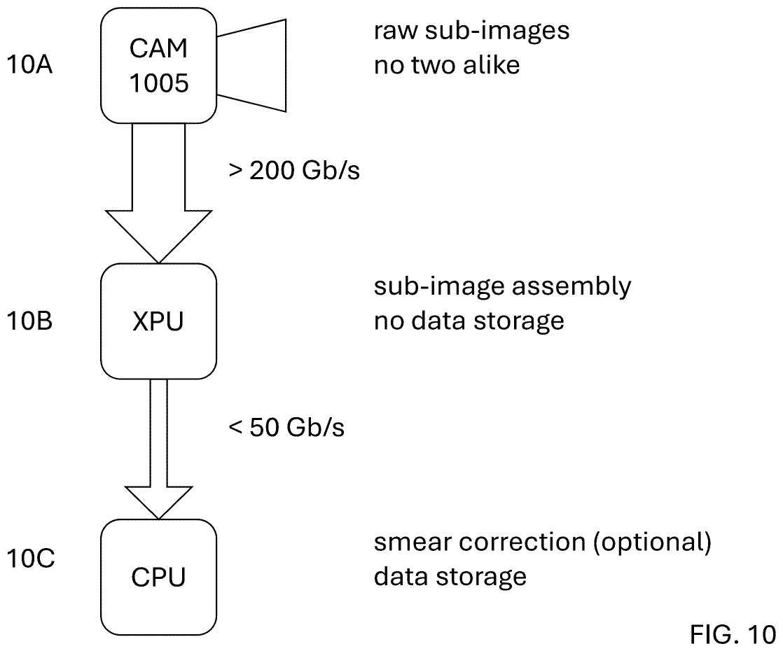

FIG. 10 schematically illustrates an implementation of a data processing aspect of the system. Pursuant to operation 10A, the camera 1005 may generate one or more sub-images, which may be at a rate that is too fast for a general purpose computer to correlate and assemble. The system includes specialized hardware is connected directly to the camera image sensors. The specialized hardware correlates, aligns and averages sub-images. In operation 10B, the specialized hardware (such as a field programmable gate array (FPGA), graphics processing unit (GPU), other custom application specific integrated circuit (ASIC), or a chip specifically designed for the image assembly task) is used to correlate and assemble the sub-images. Furthermore, the sub-images represent so much data that they may not be stored in some situations. The sub-images are processed in a real-time pipeline and discarded. In operation 10C, smear correction, if desired, is done on the assembled “final” image. A regular computer CPU can handle this operation. The specialized hardware is configured to accept the sub-images at a data rate greater than 200 Gb/s, process the sub-images, and output the sub-images at a data rate less than 50 Gb/s.

### Example Computing System

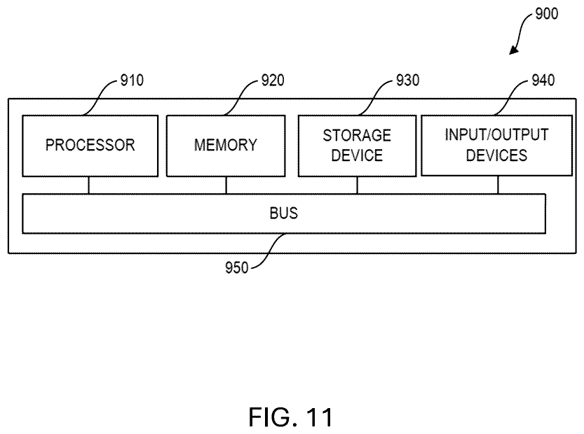

FIG. 11 depicts a block diagram illustrating an example of a computing system 900 consistent with implementations of the current subject matter. The computing system 900 may implement processes and methods described herein. The system can include or be coupled to an imaging system, an interactive user interface, and/or an inputs program to receive and manipulate physical, biometric, biomechanical, material and mechanical information and data. The system 900 can be mechanically and/or communicatively coupled to or include an imaging device such as a CT or MR device or any other imaging system. The system 900 may mechanically and/or communicatively coupled to or include a syringe system as described herein.

As shown in FIG. 11, the computing system 900 can include a processor 910, a memory 920, a storage device 930, and input/output device 940. The processor 910, the memory 920, the storage device 930, and the input/output device 940 can be interconnected via a system bus 950. The processor 910 is capable of processing instructions for execution within the computing system 900. Such executed instructions can implement one or more components of, for example, VESA, 3D-ID AI, and/or MP Tool. In some implementations of the current subject matter, the processor 910 can be a single-threaded processor. Alternately, the processor 910 can be a multi-threaded processor. The processor 910 is capable of processing instructions stored in the memory 920 and/or on the storage device 930 to display graphical information for a user interface provided via the input/output device 940.

The memory 920 is a computer readable medium such as volatile or non-volatile that stores information within the computing system 900. The memory 920 can store data structures representing configuration object databases, for example. The storage device 930 is capable of providing persistent storage for the computing system 900. The storage device 930 can be a floppy disk device, a digital cloud, a hard disk device, an optical disk device, or a tape device, or other suitable persistent storage means. The input/output device 940 provides input/output operations for the computing system 900. In some implementations of the current subject matter, the input/output device 940 includes a keyboard and/or pointing device. In various implementations, the input/output device 940 includes a display unit for displaying graphical user interfaces.

According to some implementations of the current subject matter, the input/output device 940 can provide input/output operations for a network device. For example, the input/output device 940 can include Ethernet ports or other networking ports to communicate with one or more wired and/or wireless networks, Bluetooth or digital cloud system (e.g., a local area network (LAN), a wide area network (WAN), the Internet).

In some implementations of the current subject matter, the computing system 900 can be used to execute various interactive computer software applications that can be used for organization, analysis and/or storage of data in various (e.g., tabular) format (e.g., Microsoft Excel®, and/or any other type of software). Alternatively, the computing system 900 can be used to execute any type of software application. These applications can be used to perform various functionalities, e.g., planning functionalities (e.g., generating, managing, editing of spreadsheet documents, word processing documents, and/or any other objects, etc.), computing functionalities, communications functionalities, etc. The applications can include various add-in functionalities, plug ins, or can be standalone computing products and/or functionalities. Upon activation within the applications, the functionalities can be used to generate the user interface provided via the input/output device 940. The user interface can be generated and presented to a user by the computing system 900 (e.g., on a computer screen monitor, etc.). The user interface can be integrated with other devices or virtual ecosystems.

One or more aspects or features of the subject matter described herein can be realized in digital electronic circuitry, integrated circuitry, specially designed ASICs, field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random-access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including acoustic, speech, or tactile input. Other possible input or output devices include touch screens or other touch-sensitive devices such as single or multi-point resistive or capacitive track pads, joy sticks, voice recognition hardware and software, optical scanners, optical pointers, digital image capture devices and associated interpretation software, image scanners including computer topography and magnetic resonance (MR) imaging systems and the like.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

While this specification contains many specifics, these should not be construed as limitations on the scope of an invention that is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results. Only a few examples and implementations are disclosed. Variations, modifications and enhancements to the described examples and implementations and other implementations may be made based on what is disclosed.

1. A fluorescence imaging system for imaging a plurality of fluorescent biomolecules in a sample, the system comprising:
a sample stage configured to support the sample, wherein the sample is an array of fluorescent biomolecules;
an optical system comprising a digital camera optically coupled to the sample stage, wherein the sample stage is in motion with respect to a digital camera;
a scanning fluorescence microscope coupled to the camera;
an illumination system that provides pulsed illumination;
a non-transitory memory storing executable instructions that, when executed by a processor, cause the system to:
acquire, by the digital camera, a plurality of sequentially offset, overlapping, smeared sub-images of the sample wherein all of the sub-images are obtained with the biomolecule array maintained in a common state across all the sub-images;
estimate a distance between sub-images by correlating pairs of sub-images; and
assemble and average the sub-images to obtain improved SNR in a final image.

2. The fluorescence imaging system of claim 1, wherein the biomolecule is DNA.

3. The fluorescence imaging system of claim 1, wherein the executable instructions further cause the system to acquire, by the digital camera, a second set of a plurality of sequentially offset, overlapping, smeared sub-images of the sample after biochemical processing of the biomolecule array in a flow cell and with the biomolecule in a different biochemical state.

4. The fluorescence imaging system of claim 1, wherein the system is further caused to correct the final image to remove smear.

5. The fluorescence imaging system of claim 1, wherein the system removes smear by deconvolving the smeared final image using a point spread function.

6. The fluorescence imaging system of claim 1, wherein the system does not have a time-delay integration camera or an optical image stabilization system.

7. The fluorescence imaging system of claim 1, wherein system does not synchronize the sample stage with the camera while the stage moves relative to the camera.

8. The fluorescence imaging system of claim 1, wherein each sub-image is obtained at an exposure time less than an inverse of the highest frequency of problematic vibration.

9. The fluorescence imaging system of claim 6, wherein the exposure time is 10-20 times less than the inverse of the highest frequency problematic vibration of the system.

10. The fluorescence imaging system of claim 1, wherein the system weighs less than 60-70 kg.

11. The fluorescence imaging system of claim 1, wherein the correlation of sub-images is between consecutive sub-images.

12. The fluorescence imaging system of claim 1, wherein the correlation of sub-images is between nonconsecutive sub-images.

13. The fluorescence imaging system of claim 1, wherein the optical system has a point-spread function (PSF) and wherein smear of at least one sub-image is greater than a width of the PSF by a factor of k.

14. The fluorescence imaging system of claim 13, wherein k is 1.5 to 4.

15. The fluorescence imaging system of claim 13, wherein k is 2.

16. The fluorescence imaging system of claim 13, wherein k is >1.05.

17. The fluorescence imaging system of claim 1, wherein the sub-images have insufficient SNR to be individually usable.

18. The fluorescence imaging system of claim 1, the system further comprising specialized hardware configured to accept the sub-images at a data rate greater than 200 Gb/s, process the sub-images, and output the sub-images at a data rate less than 50 Gb/s.

19. The fluorescence imaging system of claim 18, wherein the specialized hardware is a field programmable gate array (FPGA), graphics processing unit (GPU), or a custom application specific integrated circuit (ASIC).

20. A biomolecule array imaging system comprising:
a flow cell configured to contain an array of fluorescent biomolecules;
a camera, including an image sensor directly connected to dedicated processing hardware;
a pulsed illumination system;
optics, including a microscope objective lens, a tube lens and a dichroic mirror;
wherein,
the flow cell is mounted on a translation stage that moves the flow cell in a direction of motion with respect to the optics;
while the stage moves the flow cell, the pulsed illumination system illuminates a sub-section of the flow cell via the optics, and the camera captures a first set of overlapping sub-images of the flow cell via the optics;
the pulsed illumination system sets an exposure time for the sub-images;
the dedicated processing hardware estimates a distance between pairs of sub-images by cross-correlating the pairs of sub-images; and
the dedicated processing hardware aligns and averages sub-images to form a final image.

21. The system of claim 20, wherein the camera captures a second set of overlapping sub-images of the flow cell via the optics after biochemical processing in the flow cell.

22. The system of claim 20, wherein the sub-images are smeared in the direction of motion.

23. The system of claim 20, wherein the optics are characterized by a point spread function.

24. The system of claim 23, wherein the point spread function is used to deconvolve smear in the final image.

25. The system of claim 20, wherein the sub-images are characterized by a signal-to-noise ratio insufficient to render the sub-images individually useable.

26. The system of claim 20, wherein the pairs of sub-images include consecutive sub-images.

27. The system of claim 20, wherein the pairs of sub-images include non-consecutive sub-images.
